(19) United States
(12) Patent Application Publication
(10) Pub. No.: US 2026/0166101 A1
(43) Pub. Date: Jun. 18, 2026

(54) SYNBIOTIC SYSTEM OF ENCAPSULATED PROBIOTICS AND PREBIOTICS WITH TARGETED DELIVERY FUNCTION FOR COLONIZATION AND NUTRIENTS BIOCONVERSION AND THE METHOD OF MAKING THE SAME

(71) Applicant: Nano and Advanced Materials Institute Limited, Hong Kong (HK)

(72) Inventors: Joo Ann EWE, Hong Kong (HK); Chun Hay KO, Hong Kong (HK); Mu WANG, Hong Kong (HK); Yingying NG, Hong Kong (HK); Weiyi SHE, Hong Kong (HK); Yao WANG, Hong Kong (HK)

(21) Appl. No.: 18/981,636

(22) Filed: Dec. 15, 2024

(51) Int. Cl. A61K 35/747; A61K 9/48; A61K 35/00; A61K 47/61; A61K 47/64

(52) U.S. Cl. CPC A61K 35/747; A61K 9/4825; A61K 9/4833; A61K 9/485; A61K 9/4866; A61K 47/61; A61K 47/64; A61K 2035/115

(57) ABSTRACT

A synbiotic system of encapsulated probiotics and prebiotics for targeted delivery for colonization functions, comprising a probiotics core enclosed with an inner layer of protein-poly/oligosaccharide complex binder as an adhesion promoter that facilitate the attachment onto intestinal epithelial cells, and as a prebiotic promoting the growth of the enclosed probiotics upon release from the outer coating; and an outer layer of natural emulsion system coating on the synbiotic complex to minimize the involvement of water to effectively encapsulate and protect the probiotics. This system is feasible for upscale manufacturing using freeze-drying technology, and can be produced in powder form, suitable for consumption as dietary supplements as well as addition into any food in dry form. The system possesses nutrients bioconversion capability of soy isoflavones to their bioactive aglycone counterparts, as well as releases the bound bioactive phenolic acids from grains that are bioavailable for health promoting effects.

### TECHNICAL FIELD

The present invention relates to a synbiotic system. More specifically, the symbiotic system of the present invention is capable of targeted delivery for the encapsulated probiotics and prebiotics for colonization and nutrients bioconversion; and the methods of making is also provided herewith.

### BACKGROUND

The demand for functional food and dietary supplements that benefit overall wellness is growing, as consumers are more aware of healthy lifestyle for a healthy living.

Probiotics, among which, is an increasingly popular option. Often referred to or known as “good” or “beneficial” bacteria, when consumed in adequate amounts, probiotics offer a multitude of health benefits.

For example, by modulating gut microbiota, probiotics can support digestion and manage and prevent conditions like irritable bowel syndrome (IBS), diarrhea and constipation.

Probiotics can also boost and stimulate the immune system by enhancing the production of antibodies and boosting the activity of, for example, macrophages and T-lymphocytes, thereby strengthening the body to fight off infections.

There have been emerging researches showing the connection between two-way biochemical signalling between the gastrointestinal tract and the central nervous system, often referred to as the gut-brain axis, in which the gut microbiota plays a significant role in such biochemical signalling. In turn, the gut-brain axis may be linked to many disorders, including but not limited to anxiety, autism and schizophrenia, and thus the maintenance of healthy gut microbiota proves essential in alleviating such mental health conditions and symptoms.

Certain probiotics strains are also capable of bioconversion of bioactive compounds which is otherwise unachievable by the host itself, thereby facilitating digestion and immunity stimulation.

While found in fermented foods like yogurt, kefir, sauerkraut and kimchi, due to the increasingly well-known multitude of benefits, people have separated these probiotics and formulated them into dietary supplements for more direct ingestion and uptake.

However, orally administered probiotics often struggle to fully exert their health-promoting effects due to colonization resistance. Specifically, probiotics is prone to lose its viability due to its vulnerability against the strongly acidic environment in the stomach and the digestive enzymes preceding to intestinal colonization.

Even if some of the probiotics can survive through digestive transit, they may not be well-adhered to the intestinal mucosa layer because the mucoadhesive ability of probiotics is generally low inside the body.

In addition, each probiotic strain has its own specific prebiotic requirements to thrive and exert its beneficial effects on the body, therefore it is necessary to develop specific prebiotics for each probiotic strain.

To date, no existing methods in the field has been successful in promoting probiotic adherence to intestinal epithelial cells to establish targeted colonization after upper gastrointestinal digestion (acid and bile) challenges.

Most of these methods either lengthen the quantity or duration of probiotics staying in the intestine or boost probiotics activity by encapsulating prebiotics. However, empirical results showed that the growth-promotion effect of specific prebiotics varies with different probiotic species, and there is no assurance in achieving targeted colonization.

In view of the above, there is a need to develop a synbiotic system comprising probiotics and the corresponding prebiotics, with protective materials to resist the adverse environments along the digestive tract, and a binding system allowing the probiotics to retain and adhere to the intestinal epithelial mucosa for prolonged exertion of health benefits. The present invention addresses this need.

### SUMMARY OF THE INVENTION

In one aspect of the present invention, a bilayered synbiotic system for targeted delivery of probiotics, comprising an emulsion-based outer layer, a prebiotic and mucoadhesive binder inner layer and a probiotics core. The emulsion-based outer layer comprises at least one natural emulsion system; the prebiotic and mucoadhesive binder inner layer comprises an animal-based or plant-based protein hydrolysate and at least one polysaccharides or oligosaccharides; and the probiotics core comprises at least one live probiotics selected from Lactobacillus, Bifidobacterium, Lactococcus, Leuconostoc, Streptococcus, Enterococcus, Staphylococcus, Saccharomyces, Kluyveromyces or combinations thereof. Particularly, the synbiotic system comprises live probiotics in an amount of 10-30% in weight.

In one embodiment of the first aspect of the present invention, the animal-based or plant-based protein hydrolysate is selected from rice peptide, soy peptide, pea peptide, chickpea peptide, whey peptide, egg peptide, casein, milk peptide, zein, bovine serum albumin, animal collagen peptide, fish collagen peptide, or combinations thereof.

In another embodiment, the at least one polysaccharides or oligosaccharides is selected from galacto-oligosaccharides (GOS), fructo-oligosaccharides (FOS), inulin, maltodextrin, cellulose, chitin, chitosan, pectin, alginates, pullulase, konjac, arabinoxylans, or combinations thereof.

In another embodiment, the at least one natural emulsion systems is selected from cattle milk, buffalo milk, goat milk, sheep milk, camel milk, yak milk, soy milk, almond milk, coconut milk, oat milk, rice milk, barley milk, nut milk, or combinations thereof.

In a further embodiment, the bilayered synbiotic system further comprises minerals selected from chalk, talc, kaolin, zinc oxide, titanium oxide, silicon oxide, or combinations thereof.

In other embodiment, the cell adhesion of the synbiotic system to intestinal epithelial cells is at least 70%.

In yet another embodiment, the viability of the probiotics decreases no more than 2 Log CFU/g after 180 days storage at a room temperature of 20-30 degrees Celsius.

In yet other embodiment, the viable count of probiotics decreases by no more than 1 Log CFU/g after 2-hour treatment of pH 2 with pepsin under a temperature of 35-37 degrees Celsius.

In a further embodiment, the viable count of probiotics further decreases by no more than 1 Log CFU/g after 2-hour treatment of pH 6.8 with bile salt under a temperature of 35-37 degrees Celsius subsequent to the above acidic treatment.

In another embodiment, the particle size of the bilayered synbiotic system is less than 200 μm.

In other embodiment, the encapsulation efficiency of the bilayered synbiotic system is at least 90%.

In a second aspect of the present invention, a method of preparing the bilayered synbiotic system is also provided herewith. The method comprises mixing an animal-based or plant-based protein hydrolysate and at least one polysaccharides or oligosaccharides in solution and heat to 100° C. for 15 minutes to produce a protein-poly/oligosaccharide complex solution; stir-mixing the probiotics with the protein-poly/oligosaccharide complex solution to form a first solution; stir-mixing the first solution with the at least one natural emulsion system to form a second solution; and freeze-drying the second solution and grinding the crystalline to obtain the bilayered synbiotic system in the form of powder.

### BRIEF DESCRIPTION OF DRAWINGS

The appended drawings, where like reference numerals refer to identical or functionally similar elements, contain FIGS. of certain embodiments to further illustrate and clarify the above and other aspects, advantages and features of the present invention. It will be appreciated that these drawings depict embodiments of the invention and are not intended to limit its scope. The invention will be described and explained with additional specificity and detail through the use of the accompanying drawings in which:

FIG. 1 illustrates the structure of the bilayered synbiotic system of the present invention.

FIG. 2 provides a schematic diagram of the synthesis of peptide-oligosaccharides conjugates as the prebiotic and mucoadhesive binder inner layer of the synbiotic system.

FIG. 3 shows the viable counts (in logarithms) of the probiotics, L. plantarum, encapsulated in different formulations of prebiotic and mucoadhesive binder inner layers comprising a conjugate of inulin and different plant-based peptides.

FIG. 4 shows the viable counts (in logarithms) of the probiotics, L. acidophilus, encapsulated in different formulations of prebiotic and mucoadhesive binder inner layers comprising a conjugate of fructo-oligosaccharides (FOS) and different plant-based peptides.

FIG. 5 shows the cell adhesion performances of the probiotics L. plantarum encapsulated by inulin-conjugates with various plant-based peptides on mucin glycoprotein.

FIG. 6 shows the cell adhesion performances of the probiotics L. acidophilus encapsulated by FOS-conjugates with various plant-based peptides on mucin glycoprotein.

FIG. 7 shows the adhesion performances of the two probiotics encapsulated by different encapsulation formulations on Caco-2 cell monolayer.

FIG. 8 provides a schematic diagram of the production process of the freeze-dry powder of the synbiotic system of the present invention.

FIG. 9 provides the results of digestion test of pet probiotics formulation with soy milk, simulating the gastric (pH 2.0) and intestinal (pH 6.8) environments respectively.

FIG. 10 shows the sequenced feline fecal microbiome before and after the ingestion of the encapsulated probiotics of the present invention.

FIG. 11 provides the results of digestion test of human probiotics formulation with soy milk, simulating the gastric (pH 2.0) and intestinal (pH 6.8) environments respectively.

FIGS. 12A and 12B shows the test results of the bioconversion of soy isoflavones glucosides to isoflavones aglycones by the encapsulated probiotics of the present invention. FIG. 12A shows the changes in concentrations of separate species of isoflavones; and FIG. 12B shows the conglomerate changes in concentrations of glycosides and aglycones.

FIGS. 13A and 13B shows the test results of the bioconversion of tea grain powders to release free phenolic acids by the encapsulated probiotics of the present invention. FIG. 13A shows the changes in concentrations of separate species of free phenolic acids; and FIG. 13B shows the conglomerate changes in all free phenolic acids.

FIG. 14 shows the stability test results of the encapsulated probiotics of the present invention after 180-day storage in room temperature.

FIG. 15 shows schematically the bioconversion pathway of isofavones glycosides to its bioavailable aglycones form.

FIG. 16 shows schematically the bioconversion pathway of bound ferulic acid to free ferulic acid, which is bioavailable.

FIG. 17 is a schematic diagram illustrating the fermentation process of soya milk by encapsulated probiotics in soya milk, together with the process of setting up a control without probiotics, to be analyzed using HPLC.

FIG. 18 tabulates the HPLC conditions for quantifying isoflavones in soya milk samples prepared under the procedures in FIG. 17.

FIG. 19 is a chromatogram of the isoflavones glycosides and aglycones in the fermented soya milk sample prepared under the process of FIG. 17.

FIG. 20 is a schematic diagram illustrating the fermentation process of ten grains powder drinks by encapsulated probiotics, together with the process of setting up a control without probiotics, to be analyzed using HPLC.

FIG. 21 tabulates the HPLC conditions for detection of phenolic acid in the ten grains powder drinks sample prepared under the procedures in FIG. 20.

FIG. 22 is a chromatogram of the phenolic acids in the ten grains powder drinks sample prepared under the process of FIG. 20.

### DETAILED DESCRIPTION

As discussed above, functional food and dietary supplements containing probiotics with health benefits are receiving great attention from consumers, the colonization of orally administered probiotics is generally transient and limited due to their low mucoadhesive ability, as well as the inadequate number of live probiotics to pass through digestive inactivation and/or breakdown. This resulted in ineffective absorption of compounds that are non-bioactive and non-bioavailable in their precursor forms due to the limited bioconversion enzymes by the live probiotics.

The present invention provides a core-bilayer encapsulated probiotics and prebiotics comprising a probiotic core of 10-30% live probiotics materials; a prebiotic layer with the function of mucoadhesive of 15-20% protein and 36-43% poly/oligosaccharides; and a gastrointestinal digestion-resistant coating layer of 14-21% fat; and 5-7% minerals.

The protein-poly/oligosaccharide complex binder forms from the reactions between plant/animal-based protein hydrolysate and poly/oligosaccharide by heating. This complex exhibits a dual functionality, serving as both an adhesion promoter, facilitating the attachment of probiotic to intestinal epithelial cells, and a prebiotic, promoting the growth of probiotics, thereby yielding unique advantages that cannot be replicated by singular entities.

The natural emulsion system can be animal milk or plant-based milk concentrate which is rich in protein and fat. This coating material encapsulates probiotic-prebiotic complex by stirring to form a protective outer layer, thereby minimizing the involvement of water to effectively encapsulate and protect the probiotics within the coating material, which in turn exert effective viability protection during digestion, adhesion and bioconversion in gastrointestinal tract.

The encapsulated mucoadhesive-probiotics complex of the present invention is formulated with pH responsive encapsulation materials that gel at low pH to preserve the viability of probiotics against the continuous challenges from harsh gastric acid to bile salt and digestive enzymes. The encapsulated complex is also configured to disintegrate at intestinal pH to release probiotics at the target site, such that the released probiotics adhering to intestinal epithelial mucosa can colonize and perform nutrient bioconversion.

This encapsulation method produced bilayer-encapsulated probiotics in powder form, suitable for consumption as dietary supplements as well as addition into any food in dry form, and has the potential for upscale production, thereby having great industrial applicability.

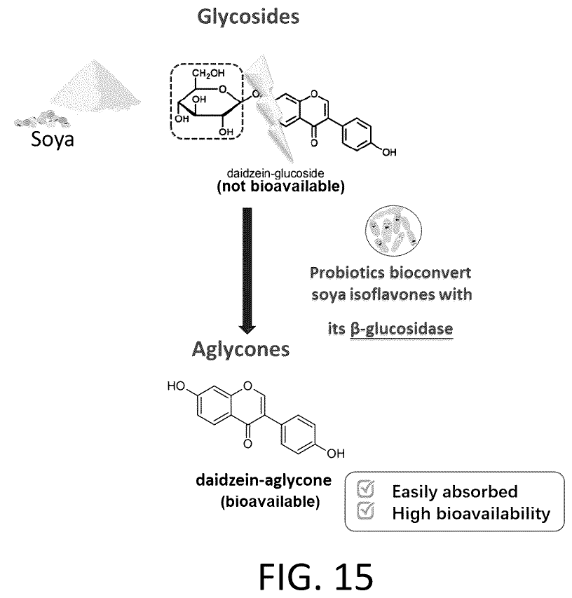

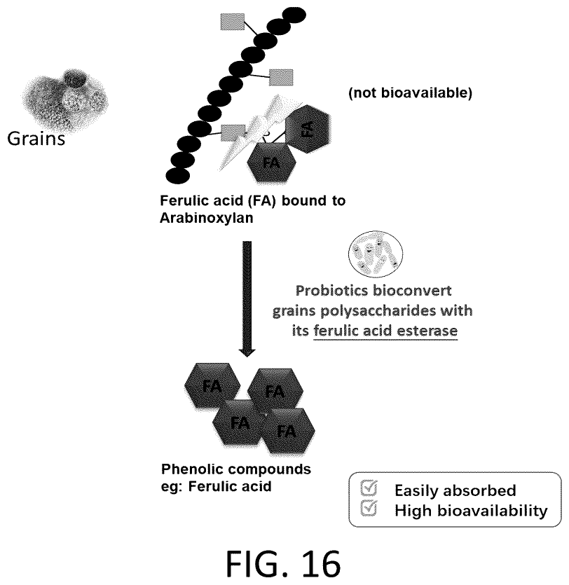

It should also be pointed out that the intestinal microflora, particularly probiotics, are found to be capable of bioconverting non-bioavailable compounds into bioavailable forms as part of their metabolism. For example, they are capable of bioconverting non-bioavailable isoflavone-glucosides into bioavailable aglycones (see FIG. 15), and non-bioavailable oligosaccharide-bound ferulic acid into free ferulic acid (see FIG. 16).

In turn, these bioactive compounds bring beneficial effects to the health of the host, including but not limited the prevention of atherosclerosis, cancer, osteoporosis and menopausal disorders.

Therefore, through the encapsulated mucoadhesive-probiotics complex of the present invention, the protection and controlled release of the probiotics is optimized such that the amount of probiotics able to reach the target site through the challenging environments of the digestive tract and undergo bioconversion is maximized.

### EXAMPLES

### Example 1-Structure of the Encapsulated Probiotics and Prebiotics with Targeted Delivery Function

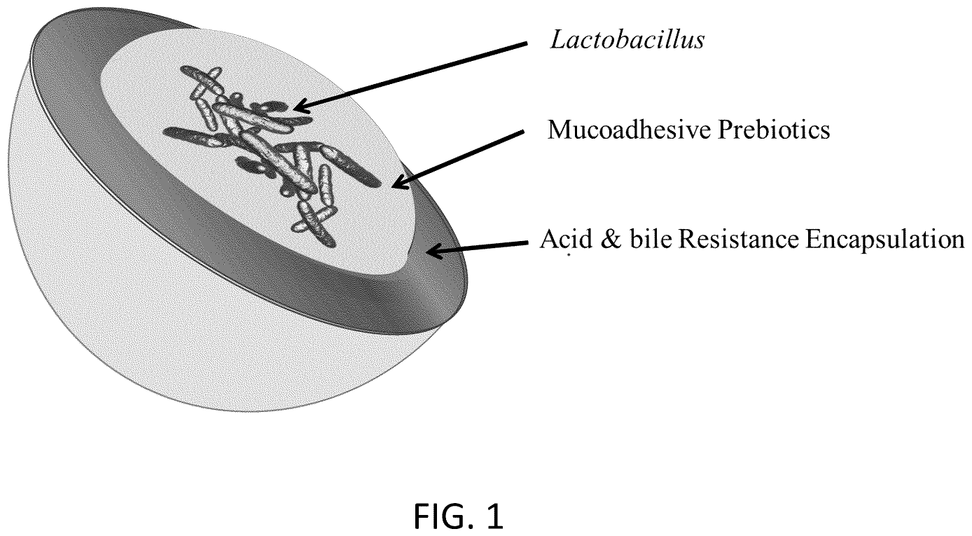

The two-layer synbiotic system of encapsulated probiotics and prebiotics with targeted delivery function for colonization is made of a core of probiotics enclosed in a prebiotic layer with the function of mucoadhesive and a coating layer with the function of gastrointestinal digestion resistance. The structure is illustrated in FIG. 1.

### Example 2-Experimental Set-Up for Growth Proliferation Test, Mucoadhesive Test and Simulated Digestion Resistance Test

### 2.1 Growth Proliferation Test

The probiotics is placed in a nutritionally deprived environment to observe the growth proliferation effects of selected prebiotics on probiotics. The prebiotics (1% w/w) were supplemented into partial nutrients (10% MRS broth) and fermented for 24 h at 37° C. Growth of probiotics in the partial nutrient's broth was used as the positive control. After 24 h, the mixture is seeded on MRS agar and incubated at 37° C. for 24-48 hours. Probiotics colonies form on the surface of the agar plate. The amount of cfu/g of sample is quantified to calculate the growth proliferation effect of the prebiotics.

### 2.2 Mucoadhesive Test on Mucin Glycoprotein

The mucoadhesive binder is coated on the selected probiotics by 15-minute mixing at room temperature and then added into 96 wells that were pre-coated with porcine gastric mucin type II to check the adhesion. Upon incubation, the wells are washed 3 times with PBS to remove the non-adhering probiotics and the adhered mucoadhesive binder-probiotics are detached from the well with Triton-X. The mixture is seeded on MRS agar and incubated at 37° C. for 24-48 hours. Probiotics colonies form on the surface of the agar plate. The amount of cfu/g of sample is quantified to calculate the mucin glycoprotein adhesion effect of the mucoadhesive binder.

### 2.3 Mucoadhesive Test on Intestinal Mucosa Cell Line

Caco-2 cells are grown in high glucose Dulbecco's Modified Eagle Medium supplemented with 1% (v/v) Penicillin/Streptomycin, 1% (v/v) Non-Essential Amino Acids Solution, and 10% (v/v) Fetal Bovine Serum (FBS). The cells are maintained in tissue culture flasks at 37° C. in a 5% CO₂ atmosphere, and sub-passed at a cell density reaching 80%˜90% confluence.

For the adhesion assay, Caco-2 cells are cultured (4×10⁵ cells per well) in 24 well tissue culture plates and grown at 37° C. with 5% CO₂ for 7 days to form the monolayer. The culture medium is changed every two days. Subsequently, the selected probiotics are cultured in MRS broth at 37° C. for overnight and after washing once with phosphate-buffered solution (PBS), the bacteria are resuspended in the formulation solutions with prebiotics and proteins as described previously at a concentration of approximately 10⁵ CFU/mL. Then, 500 μL of each bacteria suspension is added into each well of the cell monolayer and incubated for 6 hours at 37° C. The culture medium containing non-adherent bacteria is collected, and the cell monolayer is washed once with 250 μL of PBS to collect any non-adherent bacteria. 0.25% Trypsin/EDTA solution is added to detach the cells, and the process is halted by adding DMEM with 10% FBS. The adherent bacteria solutions and non-adherent bacteria solutions are separately diluted in PBS in a serial manner. The diluted solutions are plated on MRS agar plates and incubated for 48 hours in a 37° C. incubator. The adhesion to Caco-2 cells is calculated as the percentage of adherent bacteria among the total bacteria counted.

### 2.4 Sequential Simulated Digestion Resistance Test

The encapsulated probiotics are suspended in simulated gastric juice containing pepsin and sodium chloride at pH 2. The suspension is shaken at 120 rpm at 37° C. for 2 hours and then test the survivability after simulated gastric digestion by seeded on MRS ager. Subsequently, simulated intestinal juice containing bile salt and pancreatin is added into medium containing monopotassium phosphate at pH 6.8 and shaken at 120 rpm at 37° C. for another 2 hours. The survivability after simulated gastric and intestinal digestion is tested by seeded on MRS agar and incubated at 37° C. for 24-48 hours. Probiotics colonies form on the surface of the agar plate. The amount of cfu/g of sample is quantified as the viable probiotics.

### Example 3-Formulation of Growth Stimulating Prebiotics

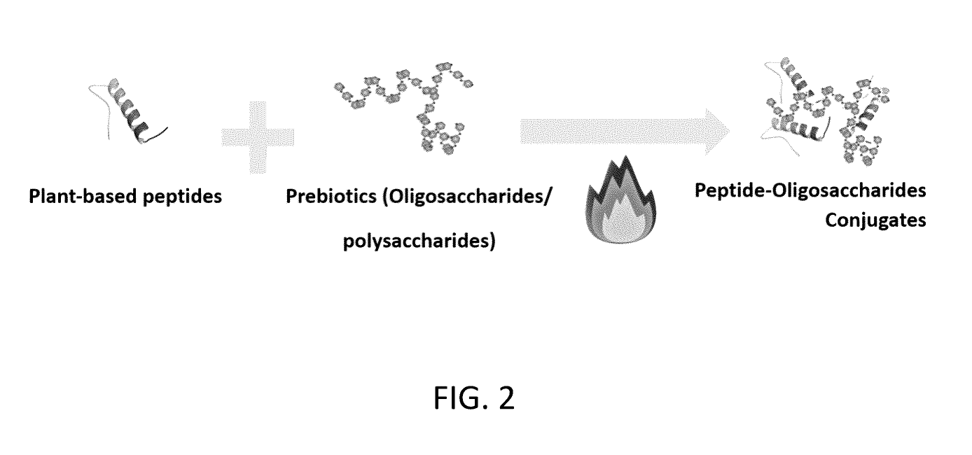

Aqueous solutions of peptides and oligosaccharides/polysaccharides are heated at 100° C. for 15 minutes to form the peptide-oligosaccharides conjugates by forming covalent bond (FIG. 2).

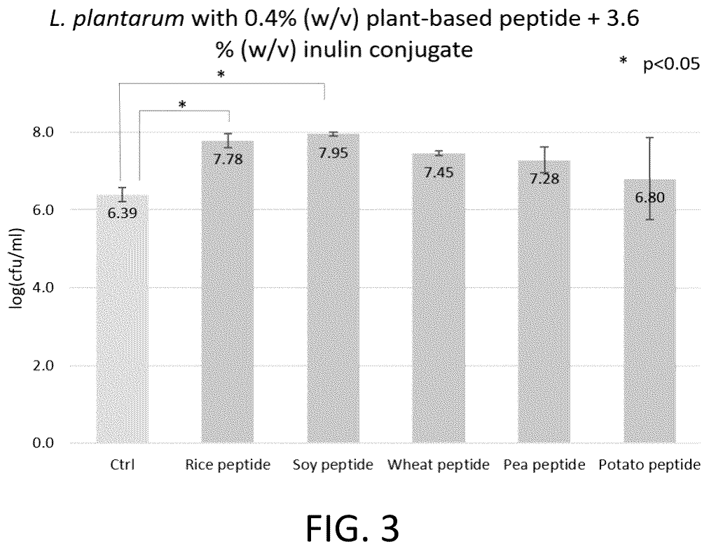

Soy peptide-inulin conjugate, rice peptide-inulin conjugate and wheat peptide-inulin conjugate as prebiotics for L. plantarum exhibit higher growth by 1.56 log cfu/ml, 1.39 log cfu/ml and 1.06 log cfu/ml than the control without probiotics, respectively (FIG. 3).

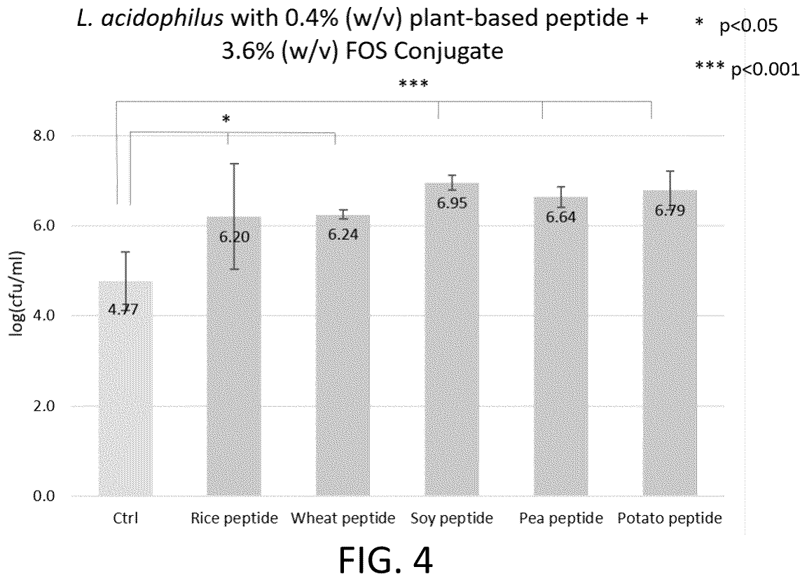

L. acidophilus supplemented with prebiotics (rice-FOS conjugate, wheat-FOS conjugate, soy-FOS conjugate, pea-FOS conjugate and potato-FOS conjugate) proliferate well by 1.43 log cfu/ml to 2.18 log cfu/ml higher than the control without prebiotics (FIG. 4).

### Example 4-Formulation of Mucoadhesive Binder for Enhanced Adhesion on Mucin Glycoprotein

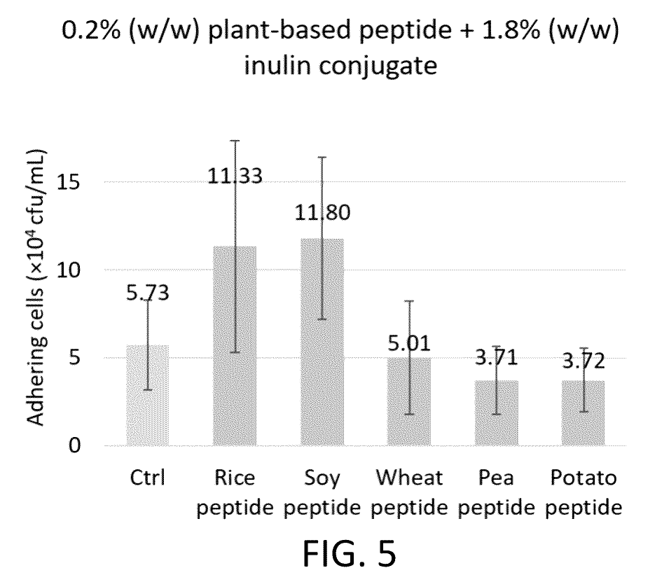

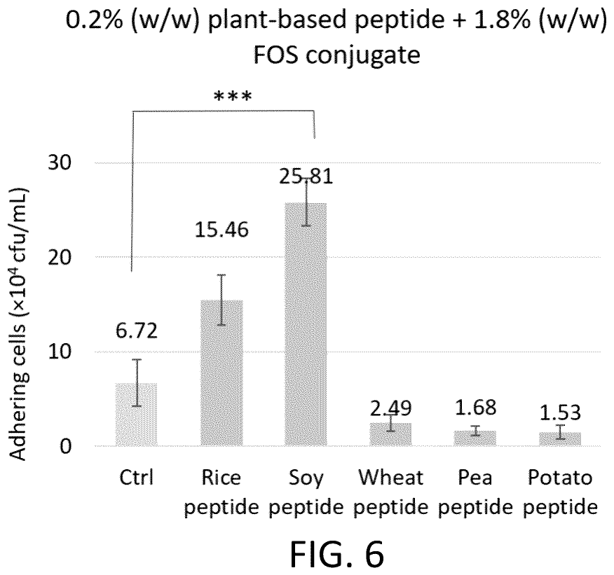

Hydrolysed protein (i.e. rice peptide, whey peptide, animal collagen peptide, fish collagen peptide) are used to form mucoadhesive binder with oligosaccharides (FOS) after boiling the mixture at 100° C. for 15 min. The conjugates are tested for adhesion on mucin glycoprotein. Inulin-conjugates made from rice peptide and soy peptide significantly enhance the adhesion of probiotics L. plantarum to the mucin by 98 and 106%; and rice-peptide-FOS and soy peptide-FOS conjugates significantly improve the L. acidophilus adhesion to mucin by 130% and 284%, respectively, compared to the control (FIGS. 5 and 6).

### Example 5-Enhanced Adhesion of Encapsulated Probiotics on Intestinal Epithelial Cells

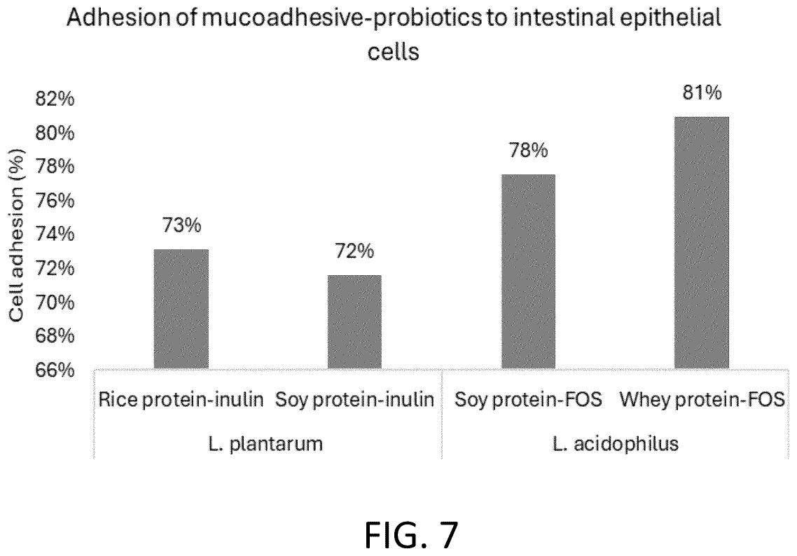

Hydrolysed protein (i.e. rice peptide, whey peptide, soy peptide) are used to form mucoadhesive binder with oligosaccharides (FOS) after boiling the mixture at 100° C. for 15 min. The developed conjugates are tested for adhesion on Caco-2 cell monolayer. L. acidophilus with mucoadhesive binder made from soy peptide/whey peptide and FOS are able to adhere to the Caco-2 cell monolayer, with a high adhesion percentage of 78 and 81%; and the adhesion ability of L. plantarum with mucoadhesive binder made from rice peptide/soy peptide and inulin are 73 and 72%, respectively (FIG. 7).

### Example 6-Encapsulation of Probiotics for Digestive Protection and Targeted Delivery

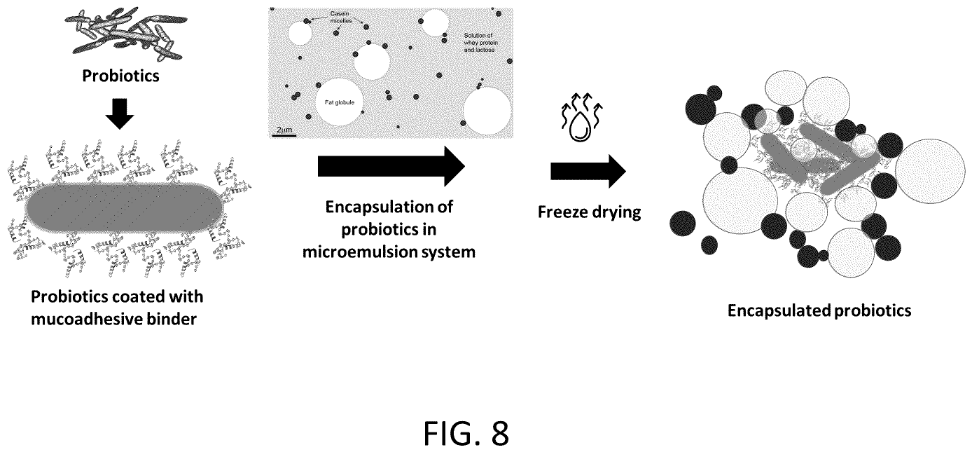

Probiotics is first enclosed by mucoadhesive binder that also serve as prebiotics in the core. Generally, after fabricating the mucoadhesive binder (soy-peptide-inulin/rice-peptide-inulin or soy-peptide-FOS), probiotics (L. plantarum or L. acidophilus) powder is added into the solution and mixed for 15 mins. Upon mixing, acid- and bile-resistant biopolymers such as macroemulsion system (e.g. milk) are added to protect the probiotics during their transit through the gastrointestinal tract. The macroemulsion system contains mainly protein (e.g. casein and whey protein for animal milk; globulin for plant-based milk), fat globules, carbohydrates and other phytochemicals or trace minerals. The fat globules are stabilized by milk fat globule membrane (MFGM) composed of polar lipids and proteins. The specific membrane structure attracted and adhered to the mucoadhesive binder of probiotics that are amphipathic, forming a protective fat globule layer. Furthermore, the milk protein in the macroemulsion system forms electrostatic interactions with the MFGM, enabling the probiotics to further embed within the fat globule-protein structure. These interactions are intensified during freeze drying, where the fat globules and proteins will be drawn towards each other as a result of casein micelle-phospholipids aggregation. These aggregated components form a protective layer by encapsulating the probiotics when in the gastric and upon reaching the intestine (FIG. 8).

### Example 7-Formulation of Probiotics Encapsulation for Pets Using Soymilk Powder

Using rice peptide-FOS-conjugate as prebiotics, formulations DSF1, DSF2 and DSF6 were formulated with different water content. Among the three concentrations of formulation tested, moderate amount of water (17.5 g in formulation DSF6) gives the greatest protection to the probiotics after freeze drying to achieve viability of 9.45±0.05 Log cfu/g and the encapsulation efficiency was 99.8%. Using similar water content, the prebiotics is modified with soy peptide-FOS-conjugates (formulation DSF5). Water content affects the viability of probiotics compared to prebiotics, where DSF5 and DSF6 achieve the viability of more than 9 Log CFU/g after encapsulation with their encapsulation efficiency more than 90% (Table 1).

TABLE 1
Formulation of probiotics encapsulation for pets using soymilk powder

| Formilation | DSF 2 | DSF1 | DSF 6 | DSF 5 |
|---|---|---|---|---|
| Probiotics (g) | 1 | 1 | 1 | 1 |
| (L. acidophilus D2: | | | | |
| L. rhanmosus GG) | | | | |
| Prebiotics & mucoadhesive | 1 (Rice | 1 (Rice | 1 (Rice | 1 (Soy |
| binder (g) | peptide) | peptide) | peptide) | peptide) |
| (plant-based peptide: FOS) | | | | |
| Soy milk powder (g) | 8 | 8 | 8 | 8 |
| CaCO₃ (g) | 1 | 1 | 1 | 1 |
| Viability (log cfu/g) | 8.94 | 8.94 | 9.45 ± 0.05 | 9.09 ± 0.04 |
| Encapsulation Efficiency | 94.4 | 94.4 | 99.81 ± 0.41 | 95.96 ± 0.48 |
| (%) | | | | |

### Example 7-Formulation of Probiotics Encapsulation for Human Using Soymilk Powder

The types of mucoadhesive binder (rice peptide-inulin-conjugates vs soy peptide-inulin-conjugates) do not affect the viability of probiotics after encapsulation (VSI3 and VSI4, Table 1), though probiotics in both formulations cannot achieve the targeted viability (9 Log cfu/g). However, lower amounts of soymilk powder used for encapsulation (6 and 7 grams vs 8 grams; VSI5 and VSI6) enhance the initial viability after encapsulation, where the viability of probiotics is 9.14±0.01 and 9.38±0.08 Log cfu/g, respectively with encapsulation efficiency of 90.12±0.14 and 92.52±0.74, respectively (Table 2). Similarly, the types of mucoadhesive binder do not affect the initial viability and encapsulation efficiency of probiotics (more than 9 Log cfu/g and more than 90% encapsulation efficiency, VSI6 and VSI7, Table 2).

TABLE 2
Formulation of probiotics encapsulation for human using soymilk powder

| Formulation | VSI 3 | VSI 4 | VSI 5 | VSI 6 | VSI 7 |
|---|---|---|---|---|---|
| Probiotics (g) | 1 | 1 | 1 | 1 | 1 |
| (L. plantarum 299v; | | | | | |
| Probio-C) | | | | | |
| Prebiotics & | 1 (Rice | 1 (Soy | 1 (Soy | 1 (Soy | 1 (Rice |
| mucoadhesive binder | peptide) | peptide) | peptide) | peptide) | peptide) |
| (g) (plant-based | | | | | |
| peptide: inulin) | | | | | |
| Soy milk powder (g) | 8 | 8 | 7 | 6 | 6 |
| CaCO₃ (g) | 1 | 1 | 1 | 1 | 1 |
| Viability (log cfu/g) | 8.93 | 8.95 | 9.14 ± 0.01 | 9.38 ± 0.08 | 9.30 ± 0.13 |
| Encapsulation | 92.4 | 92.6 | 90.12 ± 0.14 | 92.52 ± 0.74 | 91.70 ± 1.28 |
| Efficiency (%) | | | | | |

### Example 9-Digestion Test of Encapsulated Probiotics for Pets Consumption

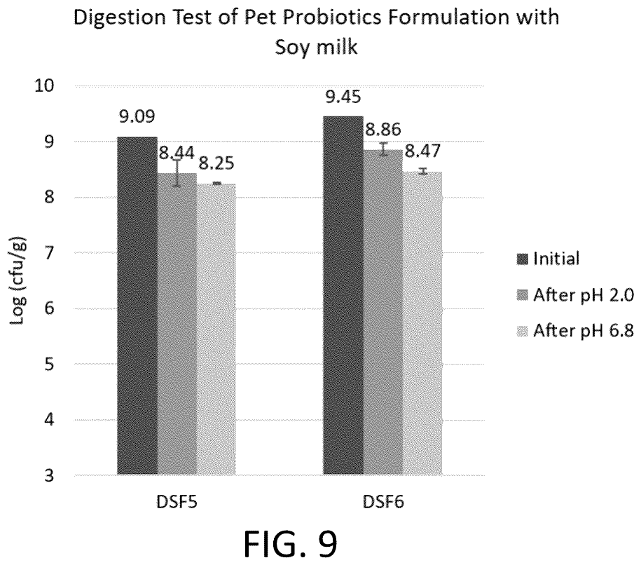

Formulations DSF5 and DSF6 are followed by digestion tests (FIG. 9). After simulated gastric acid test, there is a 0.65 Log cfu/g loss in viability for DSF5 while 0.59 Log cfu/g loss in viability for DSF6. Then, only 0.19 Log cfu/g and 0.37 Log cfu/g, respectively for DSF5 and DSF6 after simulated intestinal digestion test. Eventually, the probiotics released after all the gastrointestinal challenges are 8.25 Log cfu/g and 8.47 Log cfu/g, respectively, enabling a sufficient dose of probiotics to exert health beneficial effect to the host.

### Example 10-Effect of Probiotic Intervention on Intestinal Microbiota in Cats

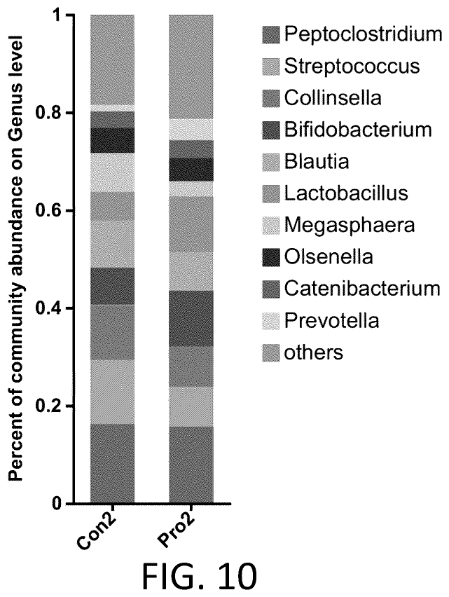

30 cats are given mucoadhesive encapsulated probiotics. Initially, the cats are acclimatized for a week and collected feces, after which probiotics intervened (15 cats) for 28 days and feces is collected afterwards. After stopping the probiotic intervention, the feces are collected again after 7 days. Samples were sequenced with intestinal microbiota 16S rRNA. 15 cats without probiotics intervention are used as control. Comparing the gut microbiome of control and probiotics groups after 28 days (FIG. 10), the abundance of Streptococcus and Collinsella genuses decreased while Bifidobacterium and Lactobacillus increased, indicating the modulation of microbiota by reducing the pathogens that causes digestive disorders and diarrhea while increasing the population of beneficial bacteria that enhance gastrointestinal health and skin health of the felines.

### Example 11-Digestion Test of Encapsulated Probiotics for Human Consumption

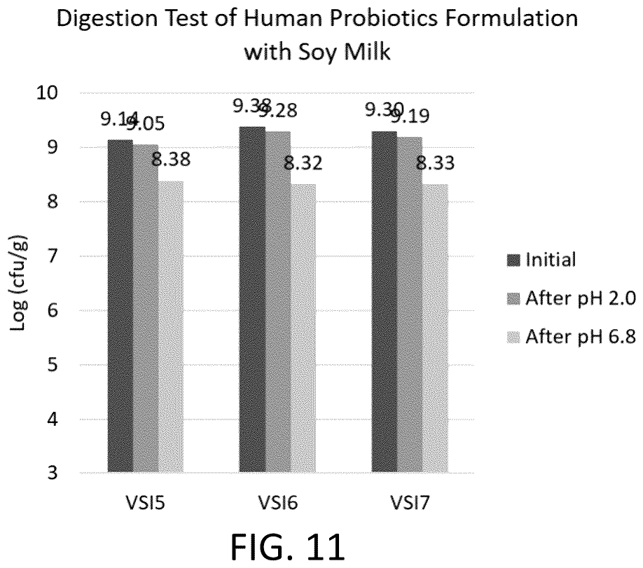

For formulations VSI5, VSI6 and VSI7, after simulated gastric acid test and intestinal digestion test, live probiotics that are released at intestines met the desirable number of live probiotics (FIG. 11). The loss in viability after acid test are 0.09, 0.10 and 0.11 Log cfu/g; after intestinal digestion were 0.67, 0.96 and 0.86 Log cfu/g, respectively. The live probiotics released in the intestine are 8.38, 8.32 and 8.33 Log cfu/g, providing sufficient live probiotics for colonization.

### Example 12-Bioconversion of Soy Isoflavones Glucosides to Aglycones by Encapsulated Mucoadhesive Probiotics

In vitro fermentation is conducted using 2% of encapsulated probiotics that have passed through digestion tests in soya milk at 37° C. for 7 h, and fermentation without probiotics is used as a control. The fermented samples are subjected to purification and the isoflavones are extracted and analyzed using HPLC.

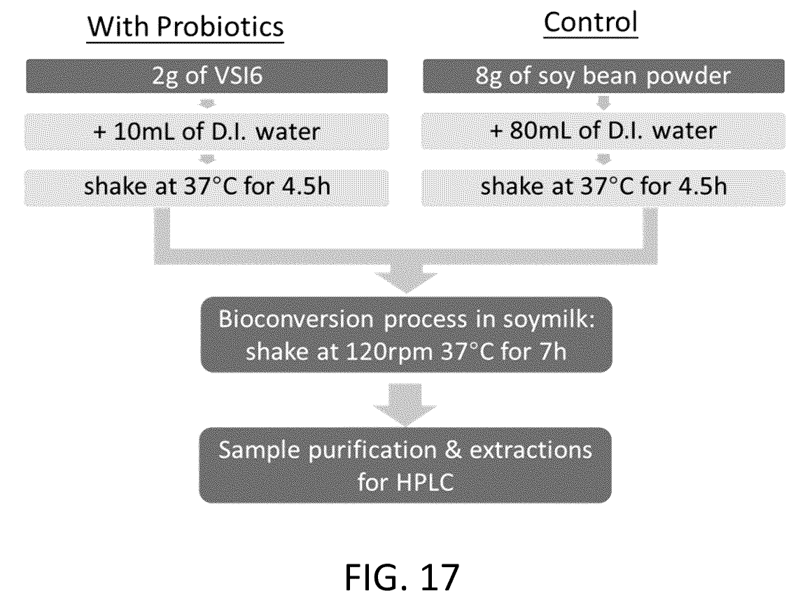

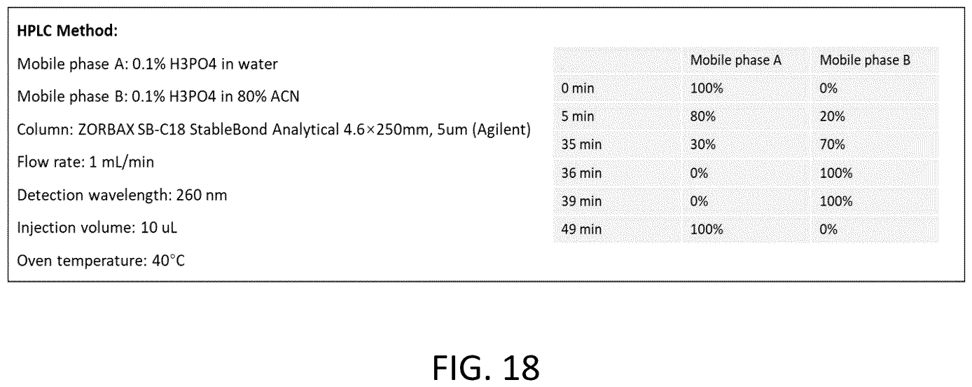

The procedures of preparing the fermented soya milk with encapsulated probiotics and the control set without probiotics are illustrated schematically in FIG. 17; and the HPLC conditions are tabulated in FIG. 18.

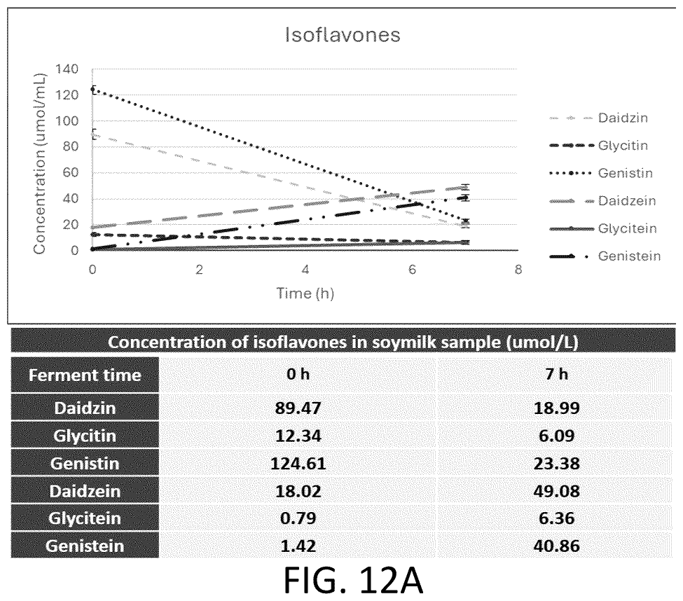

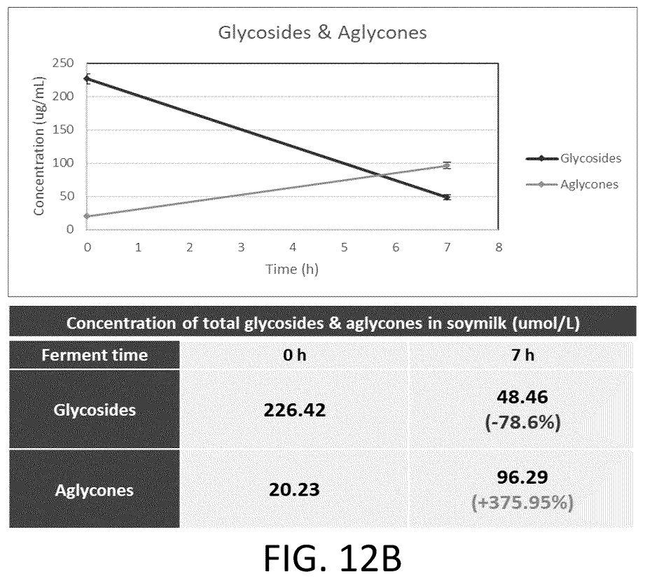

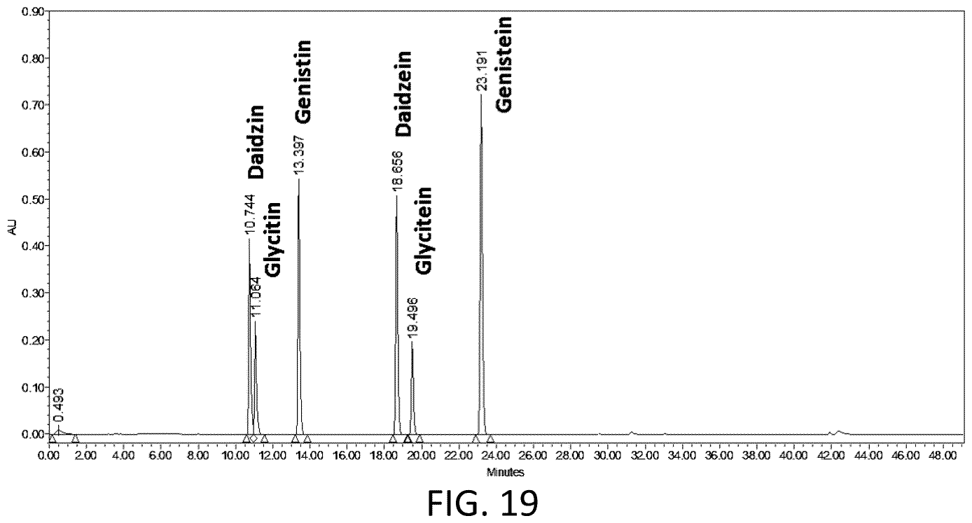

Upon 7 h fermentation, concentrations of isoflavone glycosides (daidzin, genistin and glycitin) decreased by 78.6%, while the concentrations of isoflavone aglycones (daidzein, genistein and glycitein) increased by 376% (see FIGS. 12A and 12B for the resulting concentration-time plots; and FIG. 19 for the chromatogram).

### Example 13-Bioconversion of Ten Grains Drinks by Encapsulated Mucoadhesive Probiotics

Fermentation of ten grains powder drinks is conducted using 2% activated encapsulated probiotics in ten grains powder drinks at 37° C. for 18 h, and fermentation without probiotics is used as a control.

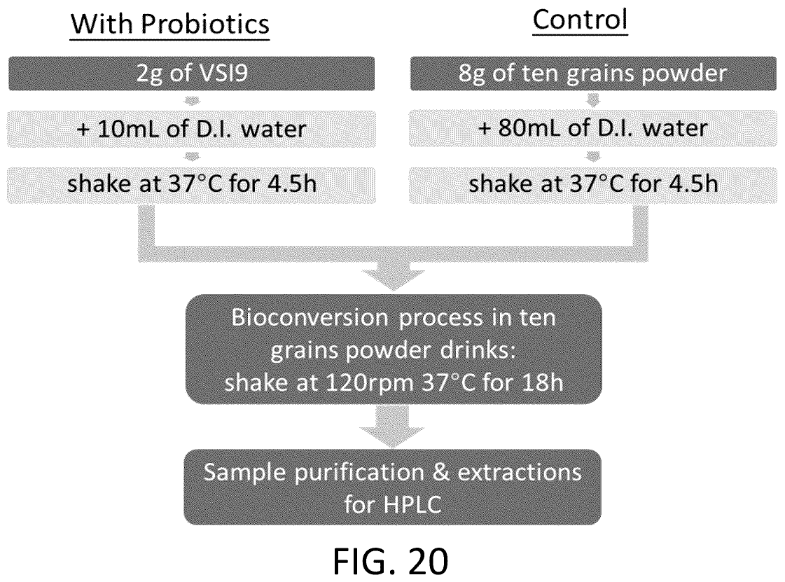

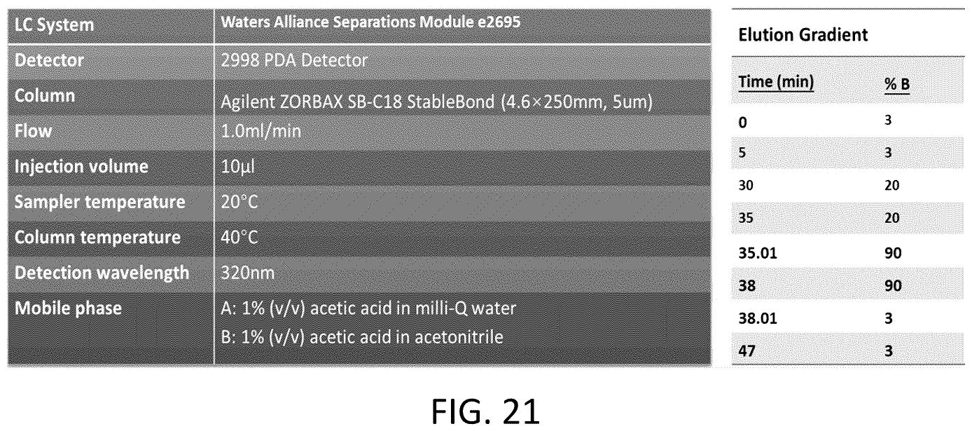

The procedures of preparing the ten grains powder drinks with encapsulated probiotics and the control set without probiotics are illustrated schematically in FIG. 20; and the HPLC conditions are tabulated in FIG. 21.

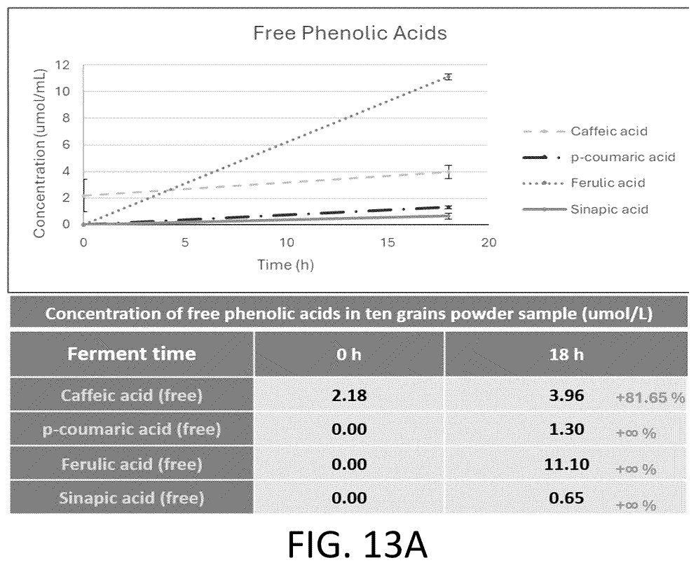

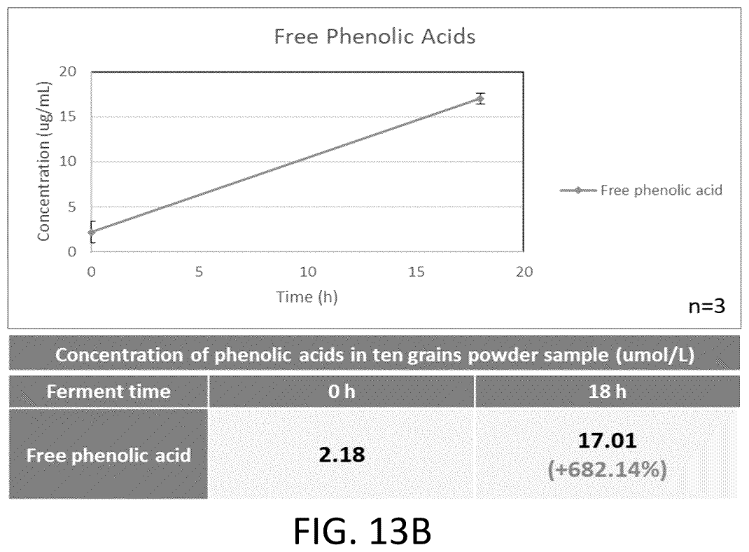

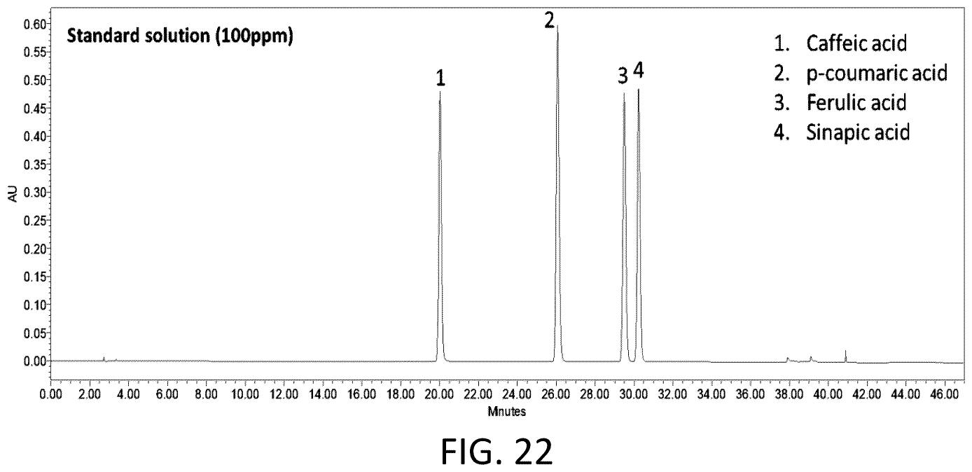

Indigenously, phenolic acids are bound to oligosaccharides in ten grains powder and are not bioavailable. Before fermentation, a very low number of free caffeic acid is detected. Other phenolic acids such as p-coumaric acid, ferulic acid and sinapic acid existed in bound form and cannot be detected. After 18 h fermentation, the bound phenolic acids are released from the bound form and the total concentrations of free phenolic acid in the sample increased by 680% compared to before fermentation (see FIGS. 13A and 13B for the resulting concentration-time plots; and FIG. 22 for the chromatogram).

### Example 14-Particle Size Distribution (Volume-Based) of Encapsulated Probiotics

The particle size of encapsulated probiotics was measured using laser diffraction combining with dynamic image analysis analyzer (SYNC-Microtrac MRB). The equivalent spherical diameter, d₅₀ of the powder is 57.28 μm (Table 3).

TABLE 3
Volume-based particle size distribution and its characteristic
diamerters of encapsulated probiotics

| | Mean Diameter (μM) |
|---|---|
| d₁₀ | 13.01 |
| d₅₀ | 57.28 |
| d₉₀ | 118.6 |

### Example 15-Viability of Encapsulated Probiotics Upon 180 Days Storage at Room Temperature

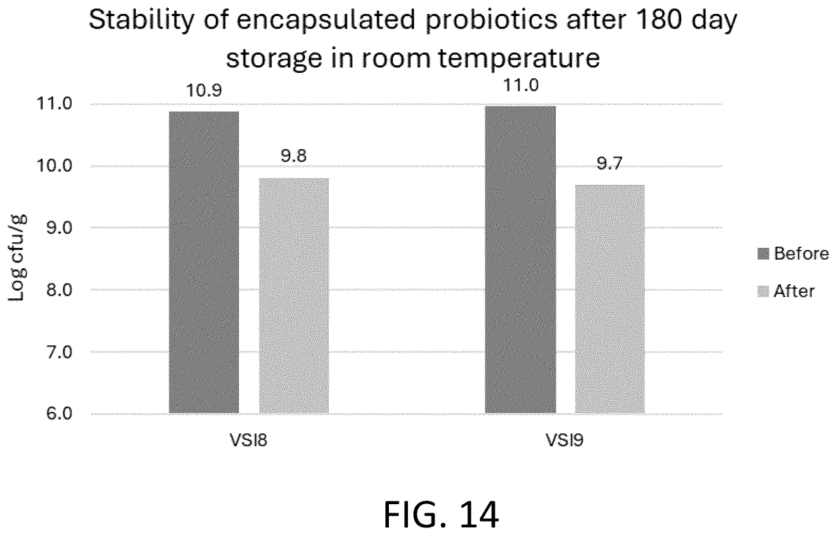

Formulation VSI8 and VSI9 using soy peptide-inulin-conjugates and rice peptide-inulin-conjugates as mucoadhesive binder/prebiotics are prepared to perform stability test upon a 180-day-storage at room temperature (Table 4). The losses of the encapsulated probiotics in viability after 180 days storage is 1.1 and 1.3 log cfu/g respectively. The viability of the encapsulated probiotics is no less than 8 log cfu/g after 180 days at 25° C. (FIG. 14).

TABLE 4
Formulation of probiotics encapsulation
for human using soymilk powder

| Ingredient | VSI 8 | VSI 9 |
|---|---|---|
| Probiotics (g) | 3 | 3 |
| (L. plantarum 299v: Probio-C) | | |
| Prebiotics and mucoadhesive binder (g) | 1 (Soy | 1 (Rice |
| (plant-based peptide: inulin) | peptide) | peptide) |
| Soy milk powder (g) | 6 | 6 |
| CaCO₃ (g) | 1 | 1 |
| Encapsulation Efficiency (%) | 98.8 | 98.7 |

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they allow for elements not explicitly recited, but exclude elements that are found in the prior art or that affect a basic or novel characteristic of the present invention.

Furthermore, throughout the specification and claims, unless the context requires otherwise, the word “include” or variations such as “includes” or “including”, will be understood to imply the inclusion of a stated integer or group of integers but not the exclusion of any other integer or group of integers.

References in the specification to “one embodiment”, “an embodiment”, “an example embodiment”, etc., indicate that the embodiment described can include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

Other definitions for selected terms used herein may be found within the detailed description of the present invention and apply throughout. Unless otherwise defined, all other technical terms used herein have the same meaning as commonly understood to one of ordinary skill in the art to which the present invention belongs.

It will be appreciated by those skilled in the art, in view of these teachings, that alternative embodiments may be implemented without undue experimentation or deviation from the spirit or scope of the invention, as set forth in the appended claims. This invention is to be limited only by the following claims, which include all such embodiments and modifications when viewed in conjunction with the above specification and accompanying drawings.

1. A bilayered synbiotic system for targeted delivery of probiotics, comprising:
an emulsion-based outer layer;
a prebiotic and mucoadhesive binder inner layer; and
a probiotics core;
wherein the emulsion-based outer layer comprises at least one natural emulsion system;
wherein the prebiotic and mucoadhesive binder inner layer comprises an animal-based or plant-based protein hydrolysate and at least one polysaccharides or oligosaccharides;
wherein the probiotics core comprises at least one live probiotics selected from Lactobacillus, Bifidobacterium, Lactococcus, Leuconostoc, Streptococcus, Enterococcus, Staphylococcus, Saccharomyces, Kluyveromyces, or combinations thereof; and
wherein the system comprises live probiotics in an amount of 10-30% in weight.

2. The bilayered synbiotic system of claim 1, wherein the animal-based or plant-based protein hydrolysate is selected from rice peptide, soy peptide, pea peptide, chickpea peptide, whey peptide, egg peptide, casein, milk peptide, zein, bovine serum albumin, animal collagen peptide, fish collagen peptide, or combinations thereof.

3. The bilayered synbiotic system of claim 1, wherein the at least one polysaccharides or oligosaccharides is selected from galacto-oligosaccharides (GOS), fructo-oligosaccharides (FOS), inulin, maltodextrin, cellulose, chitin, chitosan, pectin, alginates, konjac, arabinoxylans, or combinations thereof.

4. The bilayered synbiotic system of claim 1, wherein the at least one natural emulsion systems is selected from cattle milk, buffalo milk, goat milk, sheep milk, camel milk, yak milk, soy milk, almond milk, coconut milk, oat milk, rice milk, barley milk, nut milk, or combinations thereof.

5. The bilayered synbiotic system of claim 1, further comprising minerals selected from chalk, talc, kaolin, zinc oxide, titanium oxide, silicon oxide, or combinations thereof.

6. The bilayered synbiotic system of claim 1, wherein the cell adhesion of the synbiotic system to intestinal epithelial cells is at least 70%.

7. The bilayered synbiotic system of claim 1, wherein the viability of the probiotics decreases no more than 2 Log CFU/g after 180 days storage at a room temperature of 20-30 degrees Celsius.

8. The bilayered synbiotic system of claim 1, wherein the viable count of probiotics decreases by no more than 1 Log CFU/g after 2-hour treatment of pH 2 with pepsin under a temperature of 35-37 degrees Celsius.

9. The bilayered synbiotic system of claim 8, wherein the viable count of probiotics further decreases by no more than 1 Log CFU/g after 2-hour treatment of pH 6.8 with bile salt under a temperature of 35-37 degrees Celsius after the 2-hour treatment of pH 2 with pepsin under a temperature of 35-37 degrees Celsius.

10. The bilayered synbiotic system of claim 1, wherein the particle size of the bilayered synbiotic system is less than 200 μm.

11. The bilayered synbiotic system of claim 1, wherein the encapsulation efficiency of the bilayered synbiotic system is at least 90%.

12. A method of preparing the bilayered synbiotic system of claim 1, comprising:
mixing an animal-based or plant-based protein hydrolysate and at least one polysaccharides or oligosaccharides in solution and heat to 100° C. for 15 minutes to produce a protein-poly/oligosaccharide complex solution;
stir-mixing the probiotics with the protein-poly/oligosaccharide complex solution to form a first solution;
stir-mixing the first solution with the at least one natural emulsion system to form a second solution; and
freeze-drying the second solution and grinding the crystalline to obtain the bilayered synbiotic system in the form of powder.
